(19) United States
(12) Patent Application Publication
(10) Pub. No.: US 2013/0309298 A1
(43) Pub. Date: Nov. 21, 2013

(54) FORMULATIONS COMPRISING EXINE SHELLS

(71) Applicant: University of Hull, Hull (GB)

(72) Inventors: Stephen Lawrence ATKIN, Hull (GB); Stephen Thomas BECKETT, Wigginton (GB); Alberto DIEGO-TABOADA, Hull (GB); Grahame MACKENZIE, Hull (GB)

(21) Appl. No.: 13/928,129

(22) Filed: Jun. 26, 2013

Related U.S. Application Data
(63) Continuation of application No. 12/747,484, filed on Sep. 20, 2010.

(30) Foreign Application Priority Data
Dec. 18, 2007 (GB) 0724550.9

(51) Int. Cl. A61K 9/48; C09K 3/00; A61K 35/60

(52) U.S. Cl. CPC A61K 9/4816; A61K 35/60; A61K 9/4858; A61K 9/4866; C09K 3/00
USPC 424/452; 424/523; 514/1.1; 424/94.1; 424/93.1; 106/406

(57) ABSTRACT

A formulation containing an active substance encapsulated within an exine shell of a naturally occurring spore, together with a protective additive which is also encapsulated within the exine shell.

### CROSS-REFERENCE TO RELATED APPLICATIONS

This application is a continuation of U.S. application Ser. No. 12/747,484, filed on Sep. 20, 2010, which is a U.S. national phase entry under 35 U.S.C. §371 of International Appl. No. PCT/GB2008/004150, filed on Dec. 17, 2008, which claims priority to GB0724550.9, filed on Dec. 18, 2007, all of which are hereby incorporated herein by reference in their entirety for all purposes.

### FIELD OF THE INVENTION

This invention relates to active substance-containing formulations, their preparation and their uses.

### BACKGROUND TO THE INVENTION

It is known from WO-2005/000280 to use the exine coatings of naturally derived (typically plant) spores as delivery vehicles for pharmaceuticals and dietetic substances. These coatings can be isolated from spores by successive treatments with organic solvents, alkali and acid so as to remove the lipid, carbohydrate, protein and nucleic acid components that may be attached to or contained within the exine shell. Enzymatic methods have also been used to isolate the exine coating from other components of a spore.

Exine coatings (or shells) take the form of essentially hollow capsules which can be impregnated or filled with, or chemically or physically bound to, another substance. They are known to be chemically and physically extremely stable. According to WO-2005/000280, a pharmaceutical or dietetic active substance may be physically or chemically bound to, adsorbed on or more typically encapsulated within such a hollow exine shell. The exine/active substance combination may then be formulated—often mixed with conventional excipients, diluents or carriers and/or with release rate modifiers—for the desired mode of delivery, for example oral, buccal or pulmonary delivery.

WO-2007/012857 also discloses the use of exine shells as delivery vehicles in topical formulations. This document describes how the exine shells, despite their mechanical and chemical strength, can be caused by gentle rubbing to release a substance encapsulated within them. This makes the exine shells particularly suitable for topical delivery of substances such as cosmetics or sunscreens to surfaces such as the skin.

Sometimes, when formulating an active substance, it is necessary to protect the substance, at least temporarily, from external influences such as light, moisture or oxygen (air). This may be for the purpose of improving the storage stability of the formulation, or it may be to ensure that the formulation reaches, following its delivery to a patient, the appropriate part of the body. In particular a drug or nutraceutical which is to be delivered orally may need to be protected against the harsh acidic environment inside the stomach, if it is to reach its ultimate intended destination such as the intestine or the bloodstream. This applies for example to hydrophilic, hydrolysable and/or acid-labile substances such as proteins.

Similar protection may also be needed for volatile active substances, which might otherwise be prematurely lost through the pores of an exine delivery vehicle.

Exine shells can themselves provide a degree of protection for an encapsulated active substance, for instance from atmospheric effects such as light and/or oxygen (air), and therefore from premature degradation. The physical protection they provide can also help reduce loss of the active substance by evaporation, diffusion or leaching. It has also been found (as disclosed in WO-2007/012856) that in cases an exine shell can itself act as an antioxidant, rather than merely as a physical barrier to oxygen (air), this effect being observable even when an active substance is outside of, rather than encapsulated within, the shell.

However, exine shells are known to have nano-sized pores in their surfaces, through which it has now been found that external agents such as gastric fluid can enter a shell and attack an active substance encapsulated within it, or through which an encapsulated active substance can leach out prematurely. In particular it has been found that a hydrophilic, hydrolysable and/or acid-labile substance such as a protein or carbohydrate, encapsulated unprotected within an exine shell and delivered orally, tends to be lost fairly rapidly when exposed to gastric fluid, and might not therefore survive for long enough, in vivo, to reach the gut or the bloodstream.

This phenomenon, and the consequent need to protect an exine-encapsulated active substance which is intended to be delivered orally, is not recognised in WO-2005/000280. The document refers to the oral delivery of exine-encapsulated active substances, and to the ability of the exine shells to be absorbed into the bloodstream and there to break down so as to release an encapsulated substance. However it implies that the exine shell itself can offer sufficient protection to an encapsulated substance as it passes through the stomach. This is now known not always to be the case, in particular for pH-sensitive and/or hydrophilic materials.

Where additional protection is desired for an encapsulated active substance, both WO-2005/000280 and WO-2007/012857 advocate the use of a protective coating around the exine shell. Suitable coating materials are said to include gum Arabic, starch, shellac, gelatine and lipids such as cocoa butter or beeswax.

It can however be difficult to apply a uniform coating around a solid particle, in particular a naturally derived exine shell, the surface of which may exhibit inhomogeneities such as craters or spikes. The coating will ideally need to block all of the pores which connect the outer shell surface with its interior cavity. The coated shell will also need to be sufficiently uniform in size and shape to ensure the resultant formulation meets quality control and regulatory standards and to provide homogeneity in active substance concentration, yet the coating must still be such as to allow an appropriate release profile for the encapsulated active substance in vivo.

Moreover, the unique properties observed for exine delivery vehicles, namely, their ability to pass into the bloodstream unhindered and there to degrade so as to release an associated active substance (or at least to proceed as far as the upper or lower gastrointestinal tract before releasing the active), might be expected to be compromised by the application of an external coating of another material.

Coatings can also, inevitably, affect the bulk properties of particles such as exine shells, in particular their flowability and cohesivity, in cases having a detrimental effect on ease of formulation and/or on ease of delivery (for example, more cohesive particles may be particularly unsuitable for pulmonary delivery). Thus the application of a protective coating can significantly complicate the formulation process.

Applied coatings can also be physically and/or chemically damaged during manufacture, transit or storage, with the result that their ability to protect an encapsulated active substance can be compromised.

It would therefore be desirable to provide an alternative method for protecting an active substance which is to be delivered in or on an exine shell, which method could overcome or at least mitigate the above described problems. It is an aim of the present invention to provide novel active substance-containing formulations, in particular for oral administration and/or for taste masking, which can provide an appropriate degree of protection for the active substance whilst also helping to achieve an appropriate release profile for the substance at its intended site of action.

### STATEMENTS OF THE INVENTION

According to a first aspect of the present invention there is provided a formulation containing an active substance encapsulated within an exine shell of a naturally occurring spore, together with a protective additive which is also encapsulated within the exine shell.

The exine shell may encapsulate two or more active substances, and/or two or more protective additives.

The invention thus proposes that a protective additive be co-encapsulated with the active substance, both within the exine shell, rather than applied as a coating around the external surface of the shell. This has been found capable of providing just as effective a degree of protection for the encapsulated active substance. That such co-encapsulation is achievable is, however, far from obvious. It might have been expected, for example, that loading an exine shell with an additive as well as an active substance would result in the active substance being pushed out of the shell, or its subsequent release from the shell being compromised.

In many cases, it might also have been expected that encapsulation of a protective additive within an exine shell would be difficult to achieve. Many materials which are conventionally used as protective additives, in for example pharmaceutical or dietetic formulations, are applied in the form of a solid coating. Such coatings are typically polymeric molecules and are often too big to be able to pass through the nano-sized pores of a naturally occurring exine shell. Thus they might have been expected to deposit as coatings around the external surfaces of exine shells rather than impregnating the shells. The present inventors have nevertheless established that if such materials can be suitably solubilised, they are able to pass into an exine shell, without displacing an already encapsulated active substance, and once inside to provide a similar protective effect to that which they would have provided in the form of an external surface coating. Again it would not necessarily have been predicted that additives which normally function as solid external coatings might also provide protection when present inside an exine shell together with an active substance.

The co-encapsulation of additives with active substances can be achieved and monitored, typically, far more readily than the application of an additive coating. Thus, protection of an encapsulated active substance can be achieved, according to the invention, more quickly and hence more cheaply than using known forms of protection. It has moreover been found that an exine shell containing both active substance and additive is still suitable for oral delivery, and that it is capable of entering, and breaking down in, the bloodstream as before. In other words, the suitability of the exine shell for use as a delivery vehicle, and the release profile of the encapsulated active substance at its intended site of action, need not be compromised by the inclusion of a protective additive in accordance with the invention. Moreover the protective additive is itself protected, at least to a degree, by the outer exine shell, which can help to maintain its integrity as a protectant rather than leaving it exposed to external influences as are conventional protective coatings.

In accordance with the invention, the co-encapsulated additive can serve to protect the active substance from external influences, whether during the processes used to formulate the materials, during their storage prior to use or on their administration in vivo. In particular, the additive may be used to protect the active substance from gastric fluid when the formulation is delivered orally, thus allowing the active substance to reach its intended site of action downstream, for example the gut or the bloodstream.

Some naturally occurring spores have non-continuous exine shells: for example some exine shells may have, either inherently or as a result of a treatment process which they have undergone, micron-sized holes in their surfaces. The present invention allows the use of such exine shells as delivery vehicles for active substances, where otherwise the micropores could render them unsuitable for encapsulating the actives.

There are inherent advantages to the use of naturally occurring exine shells as delivery vehicles, as described in WO-2005/000280 (for example at pages 3 and 4 and in the paragraph spanning pages 5 and 6) and WO-2007/012857 (see pages 4 to 5). Because of its inherent non-toxicity, for instance, a spore-derived exine shell can be particularly suitable for use as a delivery vehicle in the context of formulations which are likely to come into contact with, or be ingested by, the human or animal body. The proteinaceous materials which can otherwise cause allergic reactions to pollens are preferably removed during the processes used to isolate the exine component.

Naturally occurring exine shells have been found to be readily absorbed into, and broken down in, the bloodstream, as described in WO-2005/000280, making them ideal candidates for the systemic delivery of active substances such as pharmaceuticals or nutraceuticals. They can also be of value for the topical delivery of active substances, since they have been found capable of releasing an encapsulated active on application of only moderate pressure, for example gentle rubbing, as described in WO-2007/012857, in particular at page 3.

The exine shells prepared from any given organism also tend to be very uniform in size, shape and surface properties, unlike typical synthetic encapsulating entities. There is however significant variation in spore size and shape, and in the nature of the pores in the exine shells, between different species, allowing a formulation according to the invention to be tailored dependent on the nature and desired concentration of the active substance, the site and manner of intended application, the desired active substance release rate, the likely storage conditions prior to use, etc. . . .

It can also be possible to encapsulate relatively high quantities of an active substance within even a small exine shell. The combination of high active loadings, small encapsulant size and adequate protective encapsulation is something which can be difficult to achieve using other known encapsulation techniques, and yet can be extremely useful in the context of preparing for example pharmaceutical or dietetic preparations, foods or beverages.

As described above, an exine shell is generally inert and non-toxic. Sporopollenin for example, a component of many exine shells, is one of the most resistant naturally occurring organic materials known to man, and can survive very harsh conditions of pressure, temperature and pH as well as being insoluble in most organic solvents (see G. Shaw, “The Chemistry of Sporopollenin” in Sporopollenin, J. Brooks, M. Muir, P. Van Gijzel and G. Shaw (Eds), Academic Press, London and New York, 1971, 305-348).

The ready, and often inexpensive, availability of spore exines, together with their natural origin, also make them highly suitable candidates for active substance delivery vehicles.

In the context of the present invention, the term “naturally occurring” means that a spore is produced by a living organism, whether prokaryote or eukaryote and whether plant or animal. The spore (which term includes pollen grains and also endospores of organisms such as bacteria) may for instance be derived from a plant, or from a fungus, alga or bacterium or other micro-organism.

The exine shell may be prepared from such a spore by any suitable means, as described in more detail below.

An active substance may be any substance capable of producing an effect at the site of application. It may for example be selected from pharmaceutically active substances, dietetic active substances (which includes nutraceutically active substances), foods and food ingredients, food supplements, herbicides, pesticides and pest control agents, plant treatment agents such as growth regulators, antimicrobially active substances, cosmetics (including fragrances), toiletries, disinfectants, detergents and other cleaning agents, adhesives, diagnostic agents, dyes and inks, fuels, explosives, propellants and photographic materials. In general, the present invention may be used to protect any active substance encapsulated within a naturally occurring exine shell, whether monomeric, oligomeric or polymeric and whether organic, inorganic or organometallic.

In one embodiment of the invention, the active substance is a cosmetic substance. A cosmetic substance may for example be selected from makeup products (for example foundations, powders, blushers, eye shadows, eye and lip liners, lipsticks, other skin colourings and skin paints), skin care products (for example cleansers, moisturisers, emollients, skin tonics and fresheners, exfoliating agents and rough skin removers), fragrances, perfume products, essential oils, sunscreens and other UV protective agents, self tanning agents, after-sun agents, anti-ageing agents and anti-wrinkle agents, skin lightening agents, topical insect repellants, hair removing agents, hair restoring agents and nail care products such as nail polishes or polish removers. A perfume product may comprise more than one fragrance.

In another embodiment of the invention, the active substance may be for use in a toiletry product. It may therefore be selected from soaps; detergents and other surfactants; deodorants and anti-perspirants; lubricants; fragrances; perfume products; dusting powders and talcum powders; hair care products such as shampoos, conditioners and hair dyes; and oral and dental care products such as toothpastes, mouthwashes and breath fresheners.

In yet another embodiment of the invention, the active substance is for use in a household product. It may for example be selected from disinfectants and other antimicrobial agents, fragrances, perfume products, air fresheners, insect and other pest repellants, pesticides, laundry products (e.g. washing and conditioning agents), fabric treatment agents (including dyes), cleaning agents, UV protective agents, paints and varnishes.

In a further embodiment of the invention, the active substance is a pharmaceutical or dietetic (which includes nutraceutical) active substance, which includes substances for veterinary use. It may be a pharmaceutically active substance which is suitable for topical delivery, for example selected from substances for use in treating skin or skin structure conditions (for example acne, psoriasis or eczema), wound or burn healing agents, anti-inflammatory agents, anti-irritants, antimicrobial agents (which can include antifungal and antibacterial agents), vitamins, vasodilators, topically effective antibiotics, antiseptics and agents providing skin protection against solar radiation.

More particularly, the active substance may be suitable and/or intended and/or adapted for oral delivery. It may therefore be suitable and/or intended and/or adapted for ingestion, by either humans or animals but in particular by humans.

A pharmaceutically or nutraceutically active substance may be suitable and/or intended and/or adapted for either therapeutic or prophylactic use.

In another embodiment of the invention, the active substance is a diagnostic agent, in particular one intended for oral ingestion. It may for instance be a radioactive tracer, or a magnetic tracer for use in magnetic resonance imaging. In such cases, again the protective additive may help to ensure that the co-encapsulated active substance reaches its intended delivery site. In certain situations the release profile of the active substance may itself provide diagnostic information—for example, if the protective additive is stable in acid conditions but degrades in non-acidic conditions, as described in more detail below, then a condition such as achlorhydria of the stomach, where there is no stomach acid, could cause premature release of an encapsulated diagnostic agent, detection of which could assist diagnosis of the condition.

In yet another embodiment of the invention, the active substance is a foodstuff, which includes beverages and also food and beverage ingredients. Food and beverage ingredients may include for example dietary supplements (such as vitamins and minerals, folic acid, omega-3 oils, fibre or so-called “probiotics” or “prebiotics”), flavourings, fragrances, essential oils, colourings, preservatives, stabilisers, emulsifiers or agents for altering the texture or consistency of a food product.

In particular the active substance may be selected from pharmaceutical and dietetic active substances, diagnostic agents and foodstuffs.

The active substance may comprise a volatile substance, in particular a flavouring or fragrance. A formulation according to the invention can be particularly suitable for the delivery of such substances as the co-encapsulated additive can help to inhibit release of any volatile components prior to use.

The active substance may be sensitive to one or more external influences such as heat, light, oxygen (and/or air) or water. It may be susceptible to oxidation, for example UV-induced oxidation, under ambient conditions. It may be pH-sensitive, in particular to acidic conditions.

In an embodiment of the invention, the active substance is a hydrophilic and/or hydrolysable and/or acid-labile substance, or any other substance which is at least partially degraded or otherwise altered in the presence of gastric fluid. It may for example be a proteinaceous material, which term includes proteins, peptides, oligopeptides and polypeptides. It may be a carbohydrate, which term includes mono-, di-, oligo- and polysaccharides as well as more complex carbohydrates such as gangliosides and cerebrosides; a lipid (e.g a phospholipid, terpene or carotenoid); a nucleoside, nucleotide or nucleic acid; a vitamin or co-vitamin such as ascorbic acid or vitamin B12; an essential fatty acid such as an omega-3 oil; an essential mineral or mineral-containing substance such as one containing iron, calcium, magnesium or zinc; a glyconutrient; a phytonutrient; another nutritional agent such as folic acid; or a micro-organism such as a bacterium.

Particular examples include peptides (e.g. hormones such as insulin and growth hormones such as Somatropin); enzymes (e.g. lactase and alkaline phosphatase); probiotics (e.g. Lactococcus lactis, a Gram-positive bacterium); and prebiotics (e.g. carbohydrates such as lactulose, lactitol oligofructose, inulin and galacto-oligosaccharides, tagatose, isomalto-oligosaccharides, polydextrose and maltodextrin).

The active substance may be present, within the exine shell, in a secondary fluid vehicle such as a liquid vehicle, in particular a non-aqueous and more particularly a lipid vehicle, such as an oil. The active substance may therefore be present in the form of a solution or suspension, the term “suspension” including emulsions and other multi-phase dispersions. A secondary vehicle may for example be a water-in-oil or oil-in-water-in-oil emulsion.

The active substance may itself be a synthetic substance or a naturally occurring substance. In particular it may be derived from a natural source, more particularly a plant source.

A formulation according to the invention may contain more than one active substance. Two or more such substances may for example be co-encapsulated in the same exine shell. Instead or in addition, a formulation according to the invention may comprise two or more populations of active substance-containing exine shells, each encapsulating a different active substance.

This can also enable two or more active substances to be kept separate prior to use—of value for example if they are incompatible with one another or would interact in an undesirable manner—and then released together in situ at the intended site of action.

The protective additive may be any material which is capable of protecting the active substance from an external influence, whether chemically or physically but typically by providing a physical barrier between the external influence and the active substance. The external influence may be for example heat, light, moisture, oxygen and/or air, another substance with which the active substance is incompatible or a certain pH. The external influence may in particular be a certain pH, more particularly an acidic pH. It may be a particular type of enzyme which would otherwise be capable of degrading the active substance. As an example of a protective additive which is capable of providing a barrier by chemical means to protect the active substance from an external influence there may be mentioned a substance that acts as a pH buffer.

The additive may be a material which is capable of modifying the release of the active substance from within the exine shell, typically by delaying its release until it reaches a target site. It may be a material—for example a permeation enhancer or a protease inhibitor—which can help to target the active substance to a desired location, increase the efficiency of its delivery at a desired location or by a desired mechanism and/or enhance its release profile (typically by increasing its release rate) at that location. Examples of permeation enhancers include fatty acids, bile acids, zonola occludens toxin, salicylates and EDTA, all of which might be used to enhance the permeability of active substance-loaded exine shells. Examples of protease inhibitors include sodium glycocholate, carmostat mesilate, bacitracin, chyostatin and elastinal; these might be used as additives with for example proteinaceous active substances. See for example Peppas N A, Wood K M and Blanchette J O, Expert Opinion on Biological Therapy, Volume 4, Number 6, 1 Jun. 2004: 881-887(7); Mesiha M, Plakogiannis E and Vejosoth S, “Enhanced oral absorption of insulin from desolvated fatty acid-sodium glycocholate emulsions”, Int. J Pharm 1994, 111: 213-216; and Faano A and Uzzau S, “Modulation of intestinal tight junctions by zona occludens toxin permits enteral, administration of insulin and other macromolecules in an animal model”, J. Clin. Invest., 1997, 99: 1158-1164.

The protective additive may be a material which is capable of protecting the active substance from an environment, or another substance, with which it is incompatible, prior to use of the formulation. For example, it may protect a pharmaceutically or nutraceutically active substance which is incorporated, for instance as a supplement, into another product such as a food or beverage, by reducing or preferably preventing degradation of the active substance due to the presence of other, incompatible, substances such as acids contained in the product. In such situations the protective additive may be chosen so as to allow at least partial release of the active substance following ingestion, for example in the mouth. It is to be understood that the protective additive may itself be pharmaceutically or nutraceutically active provided it is used as to protect a second pharmaceutically or nutraceutically active substance. Thus for example ibuprofen is useful as a protective additive in combination with a second pharmaceutically or nutraceutically active substance as described in greater detail below.

In an embodiment of the invention, the protective additive is a substance useable as an enteric coating, i.e. a (typically polymeric) substance capable of protecting a co-encapsulated active against conditions in the upper portions of the digestive tract. Known enteric coating materials, which may be used as protective additives in accordance with the invention, include cellulose-based coatings and acrylic-based coatings, for example those described in more detail below.

In an embodiment, the protective additive is capable of protecting the active substance from water. For example it may be used to protect an inhaled active substance in the humid environment of the lung, but allow its subsequent release on absorption into the bloodstream.

In an embodiment of the invention, the protective additive is capable of degrading—and hence releasing the co-encapsulated active substance—at or shortly before reaching the intended site of action of the active substance, but ideally remains intact—and provides its protective effects—prior to that point. Thus for example, the additive may provide a protective effect in the stomach for an orally delivered active substance, but degrade when the formulation is exposed to a more alkaline pH in the intestine, and/or when the formulation enters the bloodstream. The additive is thus preferably stable in acid conditions, including (ideally) at extremely low pHs, for example from pH 1 to 2, such as are found in the human stomach.

In an embodiment, the protective additive is capable of dissolving, becoming permeable or otherwise degrading in response to a change in pH. Suitably it is stable in acid conditions, as described above, but degrades in neutral and/or alkaline conditions, for instance at a pH of 5.5 or 6 or 7 or greater, or of 7.5 or 8 or greater—examples include anionic methylacrylate-based coating materials which are soluble above pH 5.0, 5.5, 6.0 or 7.0 (which could afford protection in stomach acid but allow active substance release in the blood following persorption); and cationic polymethylacrylates which are soluble in gastric fluid up to pH 5.0 (and could therefore be of use for example as taste masking agents). Alternatively, the additive may be stable in alkaline conditions, but degrade in acidic conditions (i.e. at a pH of less than 7).

In another embodiment, the protective additive is capable of biochemical (for example catabolic) degradation in the bloodstream. Examples of such additives include gum Arabic, gelatine, modified starch and modified dextrin. Again in this embodiment the additive is suitably stable in acid conditions, as described above.

In many situations, once the active substance has reached its intended site of action, it will need to be released from the exine shell as rapidly as possible. This can be desirable since it can allow effective active release before the loaded exine shell can be arrested and removed by white blood cells or swept out of circulation by organs such as the lungs or liver. In such cases, it may be preferred for the protective additive to be capable of rapid degradation at the intended site of action, for example on encountering an alkaline pH. Thus, additives which dissolve or otherwise degrade in this way may be preferred over those which rely for their degradation on enzymatic catabolism in the blood.

In other cases, a delayed or otherwise controlled release may be preferred even once the active substance has reached its intended destination, in which case the additive may be chosen to help achieve the desired release profile.

The additive may be a monomeric material (for example a fatty acid such as ibuprofen, cocoa butter or lauric acid), an oligomeric material or a polymeric material. It will suitably be water insoluble. Many such additives are already known for use as excipients in for example pharmaceutical formulations and food products; they are conventionally used either as protective coatings or as matrices into which an active substance may be incorporated and from which it may subsequently be released. Such matrices may be used to alter the release pattern and/or rate of the active substance.

The additive may be a substance which is either solid or semi-solid under the normal storage conditions for the formulation (typically at room temperature). It may melt at a higher temperature (for instance, body temperature) at which the active substance is intended to be released from the formulation—examples of materials that behave in this way include cocoa butter and various fatty acids.

The additive may be a material which is capable of masking, at least partially, the flavour and/or aroma of a co-encapsulated active substance.

Particularly suitable protective additives for use in the present invention include (a) acrylic-based polymers such as the poly(alkyl)acrylates or poly(alkyl cyanoacrylates); (b) cellulosic materials, in particular cellulose-based polymers such as the cellulose acetate phthalates; (c) lipids; (d) materials having a lipid component, for example a lipid side chain, in particular those derived from fatty acids as fatty acid esters or fatty acid amides; (e) polysaccharides and (f) other synthetic polymers. It will be appreciated that certain protective additives may fall within two or more of the above general classes or may contain a mixture of components which themselves fall into different categories. Thus for example cellulose itself is a polysaccharide (type e) but gives rise to the class (b) of cellulosic materials.

Examples of additives of type (a) include the poly(meth)acrylates, in particular the polymers available under the trade name Eudragit® (Evonik Industries). These are supplied for use as enteric coating materials, in particular as pharmaceutical excipients, and are known to be pH sensitive, typically being stable in acidic conditions (including at extremely low pHs such as from 1 to 2) but dissolving or becoming permeable in alkaline conditions such as are found downstream of the stomach in the gastro intestinal tract. They typically contain a plasticiser as well as the poly(meth)acrylate component, known plasticisers including fatty acids such as lauric acid, palmitic acid or myristic acid; polyols such as glycerin; organic esters such as citrate esters and dibutyl sebacate; oils such as castor oil; (poly)alkylene glycols such as polyethylene glycol; commercial plastoids such as Plastoid E 35 L (Degussa Pharma Polymers, Röhm GmbH, Darmstadt, Germany); and (see Wu et al, AAPS PharmSciTech 2001; 2(4) article 24) ibuprofen. The plasticiser can help to coalesce the polymer particles, resulting in a more complete coating and/or protective effect. It will be appreciated that a substance such as a fatty acid, for example ibuprofen may act both as a second protective agent and as a plasticiser when used with the poly(meth)acrylate component.

A wide range of Eudragit® polymers is available, each being soluble or permeable within a certain pH range. This allows selection of an appropriate polymer to target release of a protected active substance to a specific region of the GI tract. Eudragit® L-100/55 for example, as used in the examples below, is designed to dissolve at a pH of 5.5 or above, for release in the duodenum. Eudragit® L30D-55 is also insoluble at pHs lower than 5.5.

Preferred plasticisers for use with such polymers are fatty acids, in particular lauric acid, and ibuprofen.

Because poly(meth)acrylate polymers such as Eudragit® naturally dissolve or become permeable at specific pHs, rather than requiring enzymic degradation, they can be particularly suitable for use with active substances which need to be delivered into the bloodstream and/or the intestine, in particular where rapid release of the active substance is desired. A combination of two or more such polymers may be used to protect the active substance at different locations, for example one polymer affording protection as an ingested active substance passes through the mouth (typical pH around 6.5) whilst another protects it in the stomach.

Other examples of additives of type (a) are the poly(alkyl cyanoacrylates), which are preferably used in combination with a surfactant such as a polyoxyalkylene-based surfactant (e.g. those available under the trade name Poloxamer™).

Examples of additives of type (b) include the cellulose polymers such as the acetate phthalate (CAP) polymers available for example as Aquacoat® CPD (FMC BioPolymer). These too are supplied for use as enteric coatings, and typically contain a plasticiser to facilitate moulding of the polymer to an appropriate shape during coating.

Another phthalate-based enteric coating material is hydroxypropyl methyl cellulose with sodium N-(8-[2-hydroxy benzoyl]amino) caprylate (SNAC).

Other cellulosic additives include ethyl cellulose, hydroxyethyl cellulose and hydroxypropyl methyl cellulose, which may be of particular use in achieving slow release of a co-encapsulated active substance. Other materials capable of forming hydrogels may also be of use as protective additives, again suitably for slow release applications. Further examples of cellulosic additives include regenerated cellulose, cellulose acetate butyrate and hydroxypropylmethylcellulose acetate succinate.

The term “lipid” includes isoprenoid-based materials (for example materials based on terpenes and steroids) and fatty acid-based materials including fatty acids themselves and amides and esters of fatty acids (including mono-, di- and tri-glycerides and phospholipids). Certain waxes such as Carnauba wax are made up of a mixture of components but are generally described as lipids since they contain inter alia a mixture of fatty acids, long chain alcohols and fatty acid esters. Thus examples of additives of type (c) include butters and other solid fats (e.g. cocoa butter or hardened palm kernel oil); oils (e.g. cod liver oil, or terpene-based oils such as Histoclear™, limonene, deodorised orange oil and other essential oils); phospholipids (e.g. lecithin); glycolipids; lipid sulphates and sulphonates; mono-, di- and triglycerides; waxes (e.g. Carnauba wax, lanolin or beeswax). Lipid additives may be preferred for use in food products. In some cases it may be preferred, if the active substance is an oil, for the additive not to be cellulose sulphate. As further examples of additives of type (c) may be mentioned steroids, shellac and in particular ibuprofen. The term “long chain fatty acid” includes fatty acids having a C₁₁ to C₂₂ carbon chain length, for example lauric acid, myristic acid, palmitic acid, stearic acid, behenic acid, sebacic acid, undecanedioic acid, 1,10-decanedicarboxylic acid, brassylic acid, 1,12-dodecanedicarboxylic acid or 1,15-pentadecanedioic acid. They also include fatty acid-like substances such as benzoic acid, 4-isopropylbenzoic acid, palmitoyl ascorbic acid and Sulindac™. Such materials are typically capable of dissolving in higher pH environments, in particular in plasma.

Particularly preferred fatty acid additives of type (c) are lauric acid and palmitic acid, more preferably lauric acid.

Surfactants having hydrophobic side chains may also be of use as protective additives. Examples include lecithin and sucrose esters.

Examples of materials having a lipid component (type d) include lipoproteins and glycoproteins.

Examples of additives of type (e) include, cellulose, chitin, chitosan, starch, herapin and Gum Arabic. Certain materials such as Gum Arabic comprise a complex mixture of materials but may generally be classified as polysaccharides. Starch is particularly well suited for use as an additive of type (e) because of its acidity, low solubility in water and ease of introduction into the exine shell without the use of heat. Thus for example a starch solution is typically made by firstly making an emulsion in cold water and then adding the emulsion to boiling water and allowing it to cool to room temperature. A solution is then obtained which can vary in viscosity in accordance with concentration and can be introduced into the exine shell without the use of heat. If for example if starch is used as an additive with a protein active ingredient, the protein is more likely to remain in the natural active form than if it is subjected to heat. Furthermore the acidity of starch means that it is more likely to remain intact at the acid pH of the stomach, but more likely to breakdown in the blood. Starch could also be of use as a protective additive in lipid formulations such as for example cosmetics or certain types of food and beverages.

Other polymers useable as protective additives include for example the alpha-hydroxy acids and copolymers thereof, in particular poly(lactide-coglycolide) copolymers; poly(vinyl alcohols); and polysorbates, in each case suitably combined with a protease inhibitor. Further examples of other synthetic polymers suitable for use as protective additives include polyoxyalkylene-based surfactants, polymethylsiloxane, polyvinyl pyrrolidone), polyvinyl alcohol, ethylene/vinyl acetate copolymer, polyesters, polyurethanes, polycarbonates, polystyrene, polyols, polythiols, polyamines, polyethylene, polypropylene, poly(lactic acid), poly(lactic co-glycolide acid), polyglutamic acid, soyabean protein, hydrolysates and poly FA-SA (poly fumaric acid-sebacic acid).

Of the above additive, types (a) and (b) may be particularly preferred, most particularly type (a). Such additives suitably include a plasticiser, for example 0.1% w/w or greater thereof, or 1 or 5 or 10% w/w or greater thereof. They may include up to 70% w/w of a plasticiser. In another embodiment, protective additive types (a) and (d) may be preferred.

By way of example, gum Arabic and gelatin appear to be enzymatically degraded in plasma, whilst Eudragit® L-100/55, ibuprofen, lauric acid and palmitic acid dissolve in plasma at pH 7.4. These additives are therefore particularly suitable for use in oral delivery followed by transfer into the bloodstream.

Shellac, starch, beeswax and cocoa butter are also suitable for use in for example the topical or respiratory delivery of drugs or other active substances, or for the protection—prior to consumption—of active substances contained in foods and beverages (cocoa butter for example will melt, and thus release a co-encapsulated active substance, at body temperature).

In general the protective additive may be either natural or synthetic, although in some situations vegetable-derived additives may be preferred.

At least a proportion of the additive, for example 60% w/w or greater, preferably 70 or 80 or 90 or 95 or 98% w/w or greater, and more preferably substantially all, should be present inside the exine shell with the active substance. Suitably the additive is not present on the external surface of the shell, in particular not as a continuous or semi-continuous coating.

In a formulation according to the invention, the exine shell may contain two or more protective additives in addition to the active substance. Thus in an embodiment of the invention, the exine shell encapsulates both a first and a second protective additive, as described below. At least one of the first and second additives (preferably the second) may comprise an additive of type (a), suitably with a plasticiser. Both the first and the second additives may comprise an additive of type (a), again suitably with a plasticiser. The first and second additives may be the same. They are suitably added sequentially to the encapsulated active substance.

Thus, for example, two or more “layers” of a protective additive may be applied to an active substance encapsulated within an exine shell, to increase the degree of protection afforded to the active substance. Instead or in addition, layers of two or more different protective additives may be applied sequentially to the encapsulated active substance, so as to control its subsequent release in a number of distinct stages, for instance as the exine shell passes through different environments after administration to a patient. By way of example, an outer layer of a second protective additive (e.g. Eudragit™ E100) could protect the co-encapsulated active substance in the mouth (or provide a taste masking effect, or otherwise improve patient acceptability), whilst an inner layer of a first protective additive (e.g. Eudragit™ L100-55) could afford protection as the exine shell passes through the stomach, allowing the active substance to be released only subsequently for example in the intestine or into the bloodstream.

A formulation according to the invention may be suitable and/or intended and/or adapted for delivery by any appropriate route. In particular it may be suitable and/or intended and/or adapted for delivery to a living body, which may be either a plant or an animal, in particular an animal, and in the case of an animal may be either human or non-human. Such delivery may be for example oral, buccal, nasal, pulmonary, intravenous, intra-muscular, topical, transdermal, subcutaneous, intraperitoneal, vaginal, rectal or colonic. The delivery may also be via the eye or ear. More particularly the formulation may be suitable and/or intended and/or adapted for systemic delivery, more particularly for oral delivery. For the avoidance of doubt, the terms intravenous, intra muscular, transdermal, subcutaneous and intraperitoneal application include but are not limited to application by injection.

Thus the formulation of the invention may be suitable for delivery by injection. Following injection an active substance encapsulated within a pollen exine will act as a systemically circulating drug release system. The active substance will be released in the plasma as the exines are degraded and the rate of release will be dependent on the protective additive itself. For example, the protective active may slow down the degradation of the exines therefore prolonging the circulation of the exines allowing them to be a longer lasting intravenous delivery system. This is thought to occur for example in the case of heparin, and in such cases the protective additive will also be active in its own right. For example intravenous contrast agents are transient and need to be imaged as soon as they are injected. Using a formulation according to the present invention, the encapsulated contrast agent may last longer and give more intense imaging of areas of interest. It may also be possible to use the contrast agent and at a lower dose. Similarly, intravenous antibiotics currently have to be injected at a high dose because of rapid degradation. Use of a formulation according to the present invention may both protect the antibiotic from degradation and prolong the delivery. Thus use of a formulation of the invention may (1) reduce the dose that would need to be given and (2) reduce the frequency of injection. Similarly, the encapsulation and slow release of longer lasting drugs, may circumvent antibody formation that may occur with some products such as exenatide.

In some cases the formulation according to the invention may be suitable and/or intended and/or adapted for delivery to a non-living surface or region, for instance as a disinfectant.

In a formulation according to the invention, the active substance may be chemically or physically bound to, as well as encapsulated within, the exine shell. It may be only partially encapsulated within the shell, although more preferably it is entirely contained within the shell, or substantially so.

Suitable ways in which a substance may be chemically bound to an exine shell are described in WO-2005/000280, for example in the paragraph spanning pages 4 and 5, and at pages 14 to 22 and 24 to 32. They may involve chemical derivatisation of the exine shell so as to facilitate its chemical binding to the substance in question. Chemical binding may encompass covalent or other forms of chemical bond, for example hydrogen bonds, sulphide linkages, Van der Waals bonds or dative bonds.

Physical binding of an active substance to an exine shell may include for example adsorption (e.g. involving hydrophobic/hydrophilic interactions) of the substance onto a surface (whether internal or external) of the shell.

Encapsulation of an active substance means that the substance is retained within the cavities that are inherently present in the exine shell wall and/or more preferably within the central cavity defined by the shell.

An active substance may be attached to an exine shell by more than one of the above described means; for example, it may be encapsulated within the shell and also chemically bound to it, or a portion of the substance may be adsorbed onto the outer surface of the shell whilst another portion is contained inside the shell.

The above comments may also apply, mutatis mutandis, to the association between the exine shell and the protective additive.

An exine shell of a spore is the outer coating from around the naturally occurring (“raw”) spore. It may consist in part or mainly of sporopollenin, and can be isolated from the other components of the spore such as the cellulosic intine layer, and proteinaceous and nucleic acid components, as explained above. It may be of a type described in WO-2005/000280, in particular at pages 4, 8 and 9 and in Example 1.

According to the present invention, the exine shell may be derived from any suitable naturally occurring spore, whether plant or animal in origin. In this context, the term “plant” is to be construed in its broadest sense, and embraces for example mosses, fungi, algae, gymnosperms, angiosperms and pteridosperms. Moreover the term “spore” is used to encompass not only true spores such as are produced by ferns, mosses and fungi, but also pollen grains, as are produced by seed-bearing plants (spermatophytes) and also endospores of organisms such as bacteria.

Suitable organisms from which such spores may be obtained include the following, the approximate diameters of their spores being shown in the second column:

| | | |
|---|---|---|
| Bacillus subtilis | 1.2 | μm |
| Myosotis (“forget-me-not”) | 2.4-5 | μm |
| Aspergillus niger | 4 | μm |
| Penicillium | 3-5 | μm |
| Cantharellus minor | 4-6 | μm |
| Ganomerma | 5-6.5 | μm |
| Agrocybe | 10-14 | μm |
| Urtica dioica | 10-12 | μm |
| Periconia | 16-18 | μm |
| Epicoccum | 20 | μm |
| Ryegrass | 21 | μm |
| Timothy grass | 22 | μm |
| Rye | 22 | μm |
| Lycopodium clavatum | 25 | μm |
| “Lycopodium powder” | 40 | μm |
| Maize | 80 | μm |
| Hemp | 24 | μm |
| Rape hemp | 25 | μm |
| Wheat | 23 | μm |
| Abies | 125 | μm |
| Cucurbitapapo | 200 | μm |
| Cuburbita | 250 | μm. |

Of these, Lycopodium clavatum, lycopodium powder, ryegrass, rye, Timothy grass, hemp, rape, wheat and maize pollen spores may be preferred.

Other spores from which exine shells may be extracted are disclosed in the publications referred to at page 8 of WO-2005/000280.

In a formulation according to the invention, the exine shell may have a diameter (which may be determined by scanning electron microscopy) of from 1 to 300 μm, suitably from 1 to 250 μm or from 3 to 50 μm or from 15 to 40 μm. Grass pollen-derived exines, and other exine shells of approximately 20 μm diameter, might also be expected to be suitable, as may pollen exines having diameters of up to around 80 μm. For delivery into the bloodstream, exine shells having diameters of less than 40 μm, for example of 35 or 32 or even 30 μm or less, may be most suitable.

An exine shell may be obtained from a spore in known manner, for example by harsh treatment (e.g. reflux) of the spore with a combination of organic solvent and strong acid and alkali. Suitable such methods are described for instance in WO-2005/000280 (see page 10) and in the examples below. Other less severe methods may also be employed, for instance enzyme treatment (S. Gubatz, M. Rittscher, A. Meuter, A. Nagler, R. Wiermann, Grana, Suppl. 1 (1993) 12-17; K. Schultze Osthoff, R. Wiermann, J. Plant Physiol., 131 (1987) 5-15; F. Ahlers, J. Lambert, R. Wiermann, Z. Naturforsch., 54c (1999) 492-495; C. Jungfermann, F. Ahlers, M. Grote, S. Gubatz, S. Steuernagel, I. Thom, G. Wetzels and R. Wiermann, J. Plant Physiol., 151 (1997) 513-519). Alternatively, high pressure may be used to press out the internal contents of a spore through the naturally occurring pores in its outer exine layer. These methods may be used to remove proteins or carbohydrates to obtain the exine shell that retains the largely intact morphology of the original spore.

For Lycopodium clavatum, for example, the resultant exine shell may consist entirely or essentially of sporopollenin, optionally with a proportion of other materials such as chitin, glucans and/or mannans. Ideally the majority of the protein from the original spore will have been removed.

Thus, for example, the exine shell used in a formulation according to the invention will suitably contain 2% w/w or less of nitrogen, more suitably 1.5 or 1 or 0.7 or 0.6 or 0.5% w/w or less, preferably 0.4 or 0.3% w/w or less and most preferably 0.2% w/w or less. In some cases the exine shell will contain no, or substantially no (for instance less than 0.01% w/w), nitrogen.

In one embodiment of the invention, the exine shell may additionally contain all or part of the cellulose intine layer from the naturally occurring spore. This can be achieved if the spore is subjected to treatment with only organic solvent and alkali, and not with acid. Such base hydrolysis, for instance using potassium hydroxide, can ensure that proteinaceous components of the spore are removed, yet can allow at least a proportion of the original cellulosic intine to survive.

In one embodiment of the invention, the exine shell may be intact or substantially so. In other words, apart from the micro- or nanopores which are naturally present in the surfaces of such shells, it will provide a continuous outer wall defining an inner cavity into which an active substance and protective additive can be loaded. The exine shell may however be broken or damaged in parts; the invention can thus in certain cases embrace the use of a fragment of a spore-derived exine shell; in such situations, an active substance and a protective additive may be encapsulated within one or more micro- or nanopores within the structure of the exine fragment. Suitably however the exine shell is continuous over at least 50%, suitably at least 75 or 80 or 90%, of the surface area which an exine shell from the relevant species would have if intact. Thus in many cases, the present invention relates to the use of an exine shell of a naturally occurring spore rather than to a fragment of such a shell.

The exine shell may be chemically modified, either to alter its properties (for example its solubility) or to target it to an intended site of administration (for example, to render it more surface-active), or to facilitate its attachment to the active substance and/or additive. Suitable such chemical modifications, and methods for achieving them, are described in WO-2005/000280, in particular in the paragraph spanning pages 4 and 5, and at pages 14 to 22 and 24 to 32. The outside of the exine shell may for instance be modified by the (typically chemical) attachment of functional groups such as cationic and/or anionic groups (see WO-2005/000280 and also G. Shaw, M. Sykes, R. W. Humble, G. Mackenzie, D. Marsdan & E. Phelivan, Reactive Polymers, 1988, 9, 211-217), and/or functional groups which increase the affinity of the shell for a surface to which it is intended to be applied.

A formulation according to the invention may be prepared by encapsulating both the active substance and the protective additive in a suitably prepared exine shell, for instance an exine shell which has been prepared as described above.

An active substance or additive may be encapsulated within an exine shell using known techniques, again suitably as described in WO-2005/000280. Conveniently, prepared exine shells may be immersed in a solution or suspension of the relevant substance, which is then allowed to impregnate the shells, suitably followed by a drying step to remove at least some of the residual solvent(s). Where the substance to be encapsulated is a liquid, such as an oil, the prepared exine shells may simply be immersed in the liquid, which they will then absorb.

The exine shells are suitably immersed in an excess of the substance to be encapsulated within them; the shells are then suitably filled to an extent which leaves little or no void space inside them, thus maximising protection of the active substance and helping to ensure blocking of all of the nano-sized pores in the shell surfaces.

One or more penetration enhancing agents may be used, again as described in WO-2005/000280, to aid impregnation of the shell by the active substance and/or additive. A reduced or increased pressure (with respect to atmospheric pressure) may instead or in addition be used to facilitate impregnation; for example, a mixture of exine shells and an active substance and/or protective additive may be placed under vacuum in order to increase the rate of absorption of the active and/or additive by the exine shells.

A substance may be generated in situ within an exine shell, for instance from a suitable precursor substance already associated with the shell. For example, a precursor substance may be chemically or physically bound to, or encapsulated within, an exine shell, which is then contacted with a reactant substance which reacts with the precursor to generate the desired active substance or additive. Such a method may be used to associate an exine shell with an insoluble active substance or additive, starting from soluble precursor and reactant substances.

The active substance and the additive may be encapsulated within the exine shell either simultaneously or sequentially. In the former case, the active substance and additive may be mixed together, if necessary in an appropriate solvent system, and the mixture then encapsulated within the exine shell for instance using the immersion technique described above. In the latter case, the exine shell may be impregnated firstly with the active substance or a solution or suspension thereof, and secondly with the protective additive or a solution or suspension thereof, if necessary with a drying step between the two impregnation steps.

It may be preferred for the active substance to be encapsulated before the additive, as this may serve to increase the protective effect of the additive. It is believed that in such cases, the additive may form an at least partial protective layer around the outside of an active substance “core”, and that in cases the additive may at least partially coat the inside of the exine shell, thus blocking at least some of its pores.

The exine shell may be impregnated with a protective additive more than once. For example, it may firstly be impregnated with a mixture of the active substance and a first protective additive, followed by impregnation with a second protective additive, optionally with a drying step in between the two impregnation steps. Alternatively, the exine shell may be impregnated with the active substance, then with a first protective additive and then with a second protective additive, again with optional drying steps between successive impregnation steps. The inclusion of a second protective additive in this way can help to increase the degree of protection afforded to the co-encapsulated active substance. In all these situations, the second additive may be the same as or different to the first.

In the foregoing, a “suspension” of an active substance or additive may be a dispersion, emulsion or any other multi-phase system.

The protective additive may need to be solubilised to allow its encapsulation in the exine shell. Thus the shell may be impregnated with a solution of the additive in a suitable solvent, for example an alcohol such as ethanol, isopropanol or glycerol, or acetone.

The exine shell may be loaded with any suitable quantity of the active substance, depending on the context of intended use. A formulation according to the invention may for example contain the active substance and exine shells at a weight ratio of from 0.0001:1 to 5:1, such as from 0.001:1 to 5:1 or 0.01:1 to 5:1 or from 0.1:1 to 5:1 or 0.5:1 to 5:1. Larger exine shells may be needed in order to achieve larger active substance loadings.

The amount of the protective additive contained within the exine shell may again depend on the context, for example on the natures of the active substance and additive, and the nature and degree of protection required from the additive. The weight ratio of the active substance to the additive within the exine shell may for example be from 10:1 to 0.01:1.

The exine shell may be coated with a barrier layer, for example for further protection of an associated active substance or for taste masking purposes. The barrier layer may be such as to protect, and/or prevent release of, the encapsulated active substance and protective additive until a desired time or location is reached—for instance it may prevent release in the mouth but dissolve or otherwise degrade in the stomach. Such a barrier layer may also be of use for the delivery of volatile active substances, and/or oxygen sensitive substances.

Suitable coatings are solid or semi-solid under the normal storage conditions for the formulation (typically at room temperature) but may melt at a higher temperature (for instance, body temperature) at which they are intended to be delivered. Lipid coatings may be suitable for use in this way, examples including butters and other solid fats (e.g. cocoa butter or hardened palm kernel oil), oils (e.g. cod liver oil) and waxes (e.g. Carbauba wax or beeswax). Other potential coatings may be materials which can rupture on application of pressure, for example brittle solids such as shellac, or other materials which melt, break or otherwise change on administration so as to allow release of an encapsulated active substance. Gelatin may for example be a suitable coating material.

Other known coating excipients may be chosen depending on the desired delivery route and intended site of action (for example, coatings may be used to delay, target or otherwise control release of an active substance). Various natural or synthetic coating excipients, including oligomers and polymers, may be used to protect the co-encapsulated active substance and protective additive in a formulation according to the invention. Vegetable-derived coating materials may be preferred.

Coatings may be applied to exine shells in known fashion, for instance by spraying, rolling, panning or dipping. Coatings do not necessarily have to be continuous around the entire outer surfaces of the shells.

The present invention provides, in general terms, any formulation containing an active substance and a protective additive, both co-encapsulated within an exine shell of a naturally occurring spore, which has been prepared using a method of the type described above.

A formulation according to the invention may contain—in addition to the exine shell and co-encapsulated active substance and additive—one or more additional agents for instance selected from fluid vehicles, excipients, diluents, carriers, stabilisers, surfactants, penetration enhancers or other agents for targeting delivery of the exine shell and/or the active substance to the intended site of administration.

The formulation may for example take the form of a lotion, cream, ointment, paste, gel, foam, a hydrogel lotion, a skin patch or any other physical form known for topical administration, including for instance a formulation which is, or may be, applied to a carrier such as a sponge, swab, brush, tissue, skin patch, dressing or dental fibre or tape to facilitate its topical administration. It may take the form of a viscous or semi-viscous fluid, or of a less viscous fluid such as might be used in sprays (for example nasal sprays), drops (e.g. eye or ear drops), aerosols or mouthwashes.

The formulation may for example take the form of a suppository, a pessary or ovule for vaginal, rectal or colonic delivery. It may take the form of an inhaleable formulation comprising an inhaleable carrier for pulmonary nasal administration and it may for example take the form of a solution or suspension, an emulsion, gel or hydrogel, powder, capsule or tablet for intravenous, intra-muscular, transdermal, subcutaneous or intraperitoneal delivery.

The formulation may alternatively take the form of a powder, for example when the active substance is a makeup product such as a blusher, eye shadow or foundation colour, or when it is intended for use in a dusting powder. Exine shells can be extremely efficient at absorbing liquids, in particular lipids, to result in an effectively dry product with all of the liquid encapsulated within the shells, as demonstrated in Example 11 of WO-2007/012856. Other active substances, for example food or beverage supplements or ingredients, or pharmaceutically or nutraceutically active substances, may also be formulated as powders.

For oral delivery, the formulation may for example take the form of a tablet, capsule, a soft gel capsule, pastille, granules, an elixir, lozenge, emulsion, solution or suspension, or of a food (including an animal feed) or beverage.

Other suitable pharmaceutical and dietetic dosage forms are those disclosed in WO-2005/000280, for instance at pages 3 and 6 to 9.

A second aspect of the present invention provides a product containing a formulation according to the first aspect.

The product may for example be selected from cosmetic products; toiletries (e.g. bath products, soaps and personal care products); hair care products; nail care products; dental products such as toothpastes, dentifrices, mouthwashes and dental flosses; household products (whether for internal or external use) such as surface cleaners, disinfectants, air fresheners, pest repellants and laundry and fabric treatment products; dishwashing products; paints, inks, dyes and other colouring products; adhesive products; pharmaceutical and dietetic (which includes nutraceutical) products; food and beverage products, including food and beverage additives and ingredients; agricultural and horticultural products; fuels; explosives; propellants; and photographic materials. The product may also be a component of a construction material. Examples of construction materials include building materials, medical construction materials, automotive and aviation materials and biocomposites. Biocomposites may for example be used in the manufacture of automotive parts and human joint prostheses.

The product may in particular be suitable and/or intended and/or adapted for oral administration. It may be selected from pharmaceutical (which includes veterinary) and dietetic (which includes nutraceutical) products; food products (which includes beverages) and supplemented food products; and food additives, ingredients and supplements.

Again, the product may contain more than one formulation according to the invention, each associated with a separate active substance and additive.

A third aspect of the invention provides a method for formulating an active substance, in particular for oral delivery, the method involving (a) preparing or providing an exine shell of a naturally occurring spore; (b) encapsulating the active substance in the shell; and (c) co-encapsulating in the shell, with the active substance, a protective additive. The resultant product may thus be a formulation according to the first aspect of the invention.

Preferred features of this method may be as described above in connection with the first aspect of the invention. The active substance and additive may for example be loaded into the exine shell either together or separately; if the latter, then preferably the active substance is encapsulated in the shell before the additive.

The method may be carried out for the purpose of protecting the active substance from one or more external influences, and/or for influencing the rate and/or timing of release of the active substance from the exine shell, and/or for masking (at least partially) the flavour and/or aroma of the active substance.

A fourth aspect of the present invention provides an exine shell of a naturally occurring spore, containing a pharmaceutical or dietetic active substance and a co-encapsulated protective additive, for use as a delivery vehicle for the active substance.

A fifth aspect provides the use of an exine shell of a naturally occurring spore containing a pharmaceutical or dietetic active substance and a co-encapsulated protective additive, in the manufacture of a medicament for administration to a human or animal body.

A sixth aspect provides a method of treatment of a human or animal patient in need of a pharmaceutical or dietetic active substance, which method involves administering to the patient an exine shell of a naturally occurring spore containing a therapeutically or prophylactically effective amount of the active substance and a co-encapsulated protective additive. The method may involve administering to the patient a formulation according to the first aspect of the invention which contains a therapeutically or prophylactically effective amount of the active substance.

A seventh aspect of the invention provides the use of an exine shell of a naturally occurring spore as a delivery vehicle for an active substance and a protective additive, wherein both the active substance and the additive are co-encapsulated within the exine shell.

According to the second to the seventh aspects of the invention, the exine shell may as described above also contain a cellulosic intine material from the spore. It has been found that such exine/intine combinations can be useful delivery vehicles for a range of substances. They can be prepared by subjecting a spore to base hydrolysis, for instance using potassium hydroxide, so that although proteinaceous components of the spore are removed, at least a proportion of the original cellulosic intine layer survives. Retention of the intine has in some cases been found to alter the active substance releasing and/or antioxidant properties of the exine shell, for instance as described in WO-2007/012856 and WO-2007/012857.

Throughout the description and claims of this specification, the words “comprise” and “contain” and variations of the words, for example “comprising” and “comprises”, mean “including but not limited to”, and do not exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith.

Moreover unless stated otherwise, any feature disclosed herein may be replaced by an alternative feature serving the same or a similar purpose.

The present invention will now be described by means of the following non-limiting examples.

### EXAMPLES

The following experiments demonstrate the preparation of active substance-containing formulations according to the invention, and their ability to protect the active substance from external influences, in particular acidic conditions such as might be experienced in the stomach following oral administration.

The exine shells used were extracted from the spores of Lycopodium clavatum L. (common club moss), which can be purchased for example from Unikem, Post Apple Scientific, Fluka and Tibrewala International. Only the 25 μm spores were tested; these have a reticulated outer surface and are believed to have an exine shell approximately 1.5 μm thick.

The exine shells were isolated from other components present in the spores using the extraction procedures described below. All samples were subjected to acid hydrolysis with phosphoric acid following base hydrolysis with potassium hydroxide, in order to remove both proteinaceous and cellulosic components from the raw spores. It is anticipated that the present invention could equally well be carried out, however, using spores which have been subjected only to base hydrolysis, and which therefore comprise not only the exine shell but also a proportion of the cellulosic intine layer.

Firstly, the raw spores were suspended in acetone and stirred under reflux for 4 hours. For this, 250 g of the spores were dissolved in 750 ml of acetone, and refluxed for 4 hours in a 2 litre round bottomed flask fitted with two double surface Liebigs condensers (20 cm-4 cm). The resultant defatted spores (DFS) were then filtered (porosity grade 3) and dried overnight in air.

The defatted spores were suspended in 6% w/v aqueous potassium hydroxide and stirred under reflux (preferably between 80 and 90° C., although a temperature of between 90 and 130° C. could also be used) for 6 hours. After filtration (porosity grade 3), this operation was repeated with a fresh sample of the 6% w/v potassium hydroxide solution. Again the suspension was filtered (grade 3) and the resultant solid washed with hot water (three times) and hot ethanol (twice).

At this stage, if it is desired to produce exine shells containing a proportion of the cellulosic intine layer, the washed solid can then be refluxed in ethanol (750 ml) for 2-4 hours, filtered (grade 3) and washed with acetone (once, 300 ml) before drying overnight in air. Subsequently it should be thoroughly dried, to constant weight, by freeze drying or in an oven at 60° C., so as to yield the intine-containing exine shells.

To produce acid-hydrolysed (i.e. cellulose-free) exines, the base-hydrolysed spores, following filtration and washing with hot water and ethanol, were then suspended in 75-85% v/v ortho-phosphoric acid (750 ml), and stirred at 60° C. for 5 days. They were then filtered (porosity grade 3), and washed with water (5 times, 250 ml), 2M sodium hydroxide (once, 250 ml), water (5 times, 250 ml), and ethanol (once, 300 ml). They were then refluxed in ethanol (750 ml) for 2-4 hours, filtered (grade 3), washed with acetone (once, 300 ml) and finally dried overnight in air. Subsequently they were thoroughly dried to constant weight, by freeze drying or in an oven at 60° C., to obtain the desired cellulose-depleted exines.

The resultant exine products contained little or no nitrogen (assessed by combustion elemental analysis), indicating removal of proteins and nucleic acids and hence potentially allergenic components of the original spores. Any minute traces of remaining protein would in any case have been denatured by the aggressive treatments applied to the spores. The treated exines were observed by scanning electron microscopy of microtome sections and confocal electron microscopy to be essentially hollow capsules, free of the original inner sporoplasm.

In the following examples, UV visible (UV-vis) spectroscopic measurements were taken at a wavelength of 280 nm for detection of the protein used in Example 1, 220 nm for the protein used in Example 12 and 285 nm for ascorbic acid.

### Example 1

### Gum Arabic as a Protective Additive

In this example a protein of relative molecular mass (RMM) ca. 5000 was co-encapsulated in 25 μm exines with gum Arabic as the protective additive, using the following procedure.

A solution of 62.1 mg of the protein in 0.6 ml of water, containing a few drops of ethanol as a penetration enhancer, was added dropwise to 286 mg of the prepared exines, with gentle stirring. This mixture was left under vacuum for an hour, and then dried over P₂O₅ to constant weight.

A solution of 306 mg of gum Arabic in 0.8 ml of water, again with a few drops of ethanol, was then added slowly to the protein-loaded exines, with gentle stirring. The mixture was left under vacuum for an hour before being dried over P₂O₅ to constant weight. The resultant exine shells contained 94.9 mg of protein per gram of sample.

Aliquots of this sample were treated, at room temperature, with simulated gastric fluid (SGF) containing NaCl (2 g/l) and having a pH of 1.5 adjusted with 2 M HCl. The amount of protein retained in the exines was measured every 15 minutes by UV-vis spectroscopy. After 45 minutes, 80% w/w of the protein was found to remain in the exines. In contrast in the absence of the gum Arabic protective additive, 85% w/w of the protein was released (i.e. 15% w/w of the protein was retained) after 15 minutes'exposure to the SGF under the same conditions. Thus, the co-encapsulated gum Arabic was able to retain 80% w/w of the protein in the exine shells, providing a degree of protection for it against the SGF. This effect could be utilised, in vivo, to reduce degradation of an orally administered protein formulation in the stomach prior to reaching its intended site of action, for example the bloodstream or lower intestinal tract.

### Example 2

### Gelatine as a Protective Additive

The protein used in Example 1 was co-encapsulated in 25 μm exine shells with gelatine as a protective additive, using the following procedure.

A solution of 45.3 mg of the protein in 0.4 ml of water, with a few drops of ethanol as a penetration enhancer, was added dropwise to 209.6 mg of the prepared exines, with gentle stirring. The mixture was left under vacuum for an hour and then dried over P₂O₅ to constant weight. A solution of 87.4 mg of gelatine in 0.2 ml of water, with a few drops of ethanol as a penetration enhancer, was then added dropwise to the protein-loaded exines, with gentle stirring to effect homogeneity. This mixture was left under vacuum for an hour before being dried over P₂O₅ to constant weight. The resultant exines contained 133.1 mg of protein per gram of sample.

As in Example 1, aliquots of the sample were treated with simulated gastric fluid (SGF), at room temperature. The amount of protein retained in the exines was measured every 15 minutes by UV-vis spectroscopy. After 45 minutes, 79% w/w of the protein was found to have been retained by the gelatine-protected exines. Again this can be compared to the 15% w/w protein retention observed for unprotected exines after only 15 minutes (see Example 1).

Thus the co-encapsulated gelatine was able to slow the leaching out of the hydrophilic active substance from the exine shells. Again this effect could be used in vivo to help control the release of an orally administered active substance and ensure that it reached its intended destination.

### Example 3

### Cocoa Butter and Lanolin as a Protective Additive

In this example, the Example 1 protein was used as the active substance and cocoa butter and lanolin wax together as protective additives. All three components were encapsulated together into 25 μm exine shells, using the following procedure.

A solution of 83.1 mg of protein in a mixture of 0.4 ml of water and 0.4 ml of acetone was poured with stirring into a molten mixture of 407.8 mg of lanolin (Medilan™) and 358.1 mg of cocoa butter. Then, 506.8 mg of the prepared exines were added portion-wise to the mixture with gentle stirring. The resulting mixture was left under vacuum for an hour and then freeze dried to constant weight. The resultant exine shells contained 61.0 mg of protein per gram of sample.

Aliquots of the sample were then treated with SGF, as in Example 1, and the amount of protein remaining in the exines was measured after 45 minutes by UV-vis spectroscopy.

After 45 minutes in SGF, 60% w/w of the original quantity of protein remained in the exines. This illustrates that two protective additives may be used together in the present invention, i.e. that they may be co-encapsulated in exine shells with an active substance, and thereby afford protection for the active substance for instance in an acidic environment.

### Example 4

### Ibuprofen as a Protective Additive

Ibuprofen was used as a protective additive for an exine-encapsulated protein (as used in Example 1), the two being encapsulated sequentially into 25 μm exine shells.

A solution of 51.4 mg of the protein in 0.48 ml of water with a few drops of ethanol was added dropwise to 262.6 mg of the prepared exine shells, with gentle stirring. The mixture was left under vacuum for an hour and dried over P₂O₅ to constant weight. A solution of 194.2 mg of ibuprofen in 0.24 ml of ethanol was then added dropwise to the protein-loaded exines, with gentle stirring, and the mixture was left under vacuum for an hour and then dried over P₂O₅ to constant weight. The resultant exines contained 101.2 mg of the protein per gram of sample.

Aliquots of the sample were then treated with SGF at 37° C., and the amount of protein remaining in the exines was measured after 45 minutes by UV-vis spectroscopy. 34% w/w of the original quantity of protein was found to remain.

This example illustrates that ibuprofen may be used to protect an exine-encapsulated protein from degradation in the harsh acidic conditions encountered in the stomach following oral administration. Ibuprofen is known to be released from exines in plasma, at pH 7.4, and is thus suitable for protecting an active substance which is intended to be delivered into the bloodstream or lower intestinal tract. The encapsulating exine shell would also be degraded in the bloodstream, the exine and the ibuprofen additive thus together allowing release of the active substance only on reaching its intended destination.

### Example 5

### Eudragit® L-100/55/Lauric Acid as a Protective Additive

Here, a mixture of a polymethacrylate polymer and the plasticiser lauric acid was used as a protective additive for the Example 1 protein encapsulated in 25 μm exine shells. Two separate applications of the Eudragit® L-100/55 additive were used, so as to effect a double co-encapsulated protective layer.

A solution of 140.4 mg of the protein in 1.3 ml of water was added to a solution of 101.7 mg of Eudragit® L-100/55 in 0.65 ml of acetone-water (49:1). The mixture was stirred to afford a homogeneous emulsion, which was then added dropwise to 676.5 mg of the prepared exine shells, with gentle stirring. The mixture was left under vacuum for an hour and freeze dried to constant weight.

A solution containing a mixture of 167.7 mg of Eudragit® L-100/55 (Evonik Industries AG) and 74.6 mg of lauric acid in 1.3 ml of acetone-water (49:1) was then added dropwise to the protein-loaded exines, with gentle stirring. The mixture was left under vacuum for an hour and then freeze dried to constant weight. The resultant exines contained 120.9 mg of the protein per gram of sample.

Aliquots of this sample were then treated with SGF as in Example 4, and the amount of protein remaining in the exines was measured every 15 minutes by UV-vis spectroscopy. After 45 minutes in the SGF, 94% w/w of the original quantity of protein remained in the exines. This demonstrates that the Eudragit® L-100/55/lauric acid mixture can provide extremely effective protection for the encapsulated protein against gastric fluid. This particular formulation will release the active ingredient at a pH of 7.4.

Aliquots of the sample were also treated with phosphate buffer saline (PBS) and the amount of protein remaining in the exines was measured every 15 minutes as above. After 15 minutes in the PBS, 40% w/w of the original quantity of protein remained in the exines, and 10% w/w remained after 45 minutes. Thus Eudragit® L-100/55 and lauric acid may be used together to protect active substances which are intended for oral delivery to the bloodstream or lower intestinal tract.

In all of the above examples, encapsulation of the additive (as opposed to its deposition as a coating on the external surfaces of the exines) was confirmed by scanning electron microscopy (SEM), and with confocal microscopy when the contents were fluorescent as in Example 3.

### Example 6

### Eudragit® L-100/55/Lauric Acid as a Protective Additive

A 1:2 mixture of a polymethacrylate polymer and the plasticiser lauric acid was used as a protective additive for the Example 1 protein encapsulated in 25 μm exine shells.

A solution of 72.4 mg of the protein dissolved in 0.7 ml of water and 0.17 ml of ethanol was added dropwise to 319.6 mg of the exine shells, with gentle stirring, and the mixture left under vacuum for an hour. The sample was dried over P₂O₅ to constant weight. A solution containing a mixture of 63.1 mg of Eudragit® L-100/55 and 191.1 mg of lauric acid in 0.5 ml of ethanol was then added dropwise to the protein-loaded exines, with gentle stirring, and the mixture was left under vacuum for an hour and then dried to constant weight. The resultant exines contained 112.0 mg of the protein per gram of sample.

Aliquots of the sample were then treated with SGF, as in Example 4, and the amount of protein remaining in the exines was measured every 15 minutes by UV-vis spectroscopy. After 45 minutes in the SGF, 44% w/w of the original quantity of protein remained in the exines. The protein was released when treated with PBS as in Example 5.

### Example 7

### Eudragit® L-100/55/Lauric Acid as a Protective Additive

A 1:1 mixture of a polymethacrylate polymer and the plasticiser lauric acid was used as a protective additive for the Example 1 protein encapsulated in 25 μm exine shells, as in Example 6.

A solution of 79.8 mg of the protein in 0.74 ml of water and 0.18 ml of ethanol was added dropwise to 352.2 mg of the prepared exine shells, with gentle stirring, and the mixture left under vacuum for an hour. The sample was dried over P₂O₅ to constant weight. A solution containing a mixture of 119.1 mg of Eudragit® L-100/55 and 110.5 mg of lauric acid in 1 ml of ethanol was then added dropwise to the protein-loaded exines, with gentle stirring, and the mixture was left under vacuum for an hour and then dried over P₂O₅ to constant weight. The resultant exines contained 120.6 mg of the protein per gram of sample.

Aliquots of the sample were treated with SGF, as in Example 4, and the amount of protein remaining in the exines was measured every 15 minutes by UV-vis spectroscopy. After 45 minutes 81% w/w of the original quantity of protein remained in the exines. The protein was released when treated with PBS as in Example 5.

### Example 8

### Eudragit® L-100/55/Lauric Acid as a Protective Additive

A mixture of a polymethacrylate polymer and the plasticiser lauric acid was used as a protective additive for the Example 1 protein encapsulated in 25 μm exine shells. Two separate applications of the Eudragit® L-100/55 and lauric acid additive were used to effect a double co-encapsulated layer.

A solution of 71.9 mg of the protein in 0.66 ml of water and 0.17 ml of ethanol was added dropwise to 317.7 mg of the exine shells, with gentle stirring, and the mixture left under vacuum for an hour. The sample was freeze dried to constant weight. A solution containing a mixture of 62.6 mg of Eudragit® L-100/55 and 67.8 mg of lauric acid in 0.5 ml of ethanol was then added dropwise to the protein-loaded exines, with gentle stirring. The mixture was left under vacuum for an hour and then dried to constant weight. This operation was repeated with a solution of 59.5 mg of Eudragit® L-100/55 and 55.2 mg of lauric acid in 0.5 ml of ethanol, which again was added dropwise to the protein-loaded exines, with gentle stirring. The final mixture was left under vacuum for an hour and then dried to constant weight. The resultant exines contained 113.3 mg of the protein per gram of sample.

Aliquots of the sample were then treated with SGF, as in Example 4, and the amount of protein remaining in the exines was measured every 15 minutes by UV-vis spectroscopy. After 45 minutes, 86% w/w of the original quantity of protein remained in the exines. The protein was released when treated with PBS as in Example 5.

### Example 9

### Eudragit®/Lauric Acid as a Protective Additive

A 1:1 mixture of a polymethacrylate polymer and the plasticiser lauric acid was used as a protective additive for the Example 1 protein encapsulated in 25 μm exine shells. Two separate applications of the additive were made.

A solution of 64.8 mg of the protein dissolved in 0.6 ml of water and 0.3 ml of acetone was added dropwise to 344.2 mg of the prepared exine shells, with gentle stirring, and the mixture left under vacuum for an hour. The sample was freeze dried to constant weight. A solution containing a mixture of 80.8 mg of Eudragit® L-100/55 and 83.4 mg of lauric acid in 0.6 ml of acetone containing 2% water was then added dropwise to the protein-loaded exines, with gentle stirring. The mixture was left under vacuum for an hour and then freeze dried to constant weight. This operation was repeated with a solution of 80.8 mg of Eudragit® L-100/55 and 83.4 mg of lauric acid in 0.6 ml of acetone containing 2% water, which was added dropwise to the protein-loaded exines, again with gentle stirring. The mixture was left under vacuum for an hour and then dried to constant weight. The resultant exines contained 90.4 mg of the protein per gram of sample.

Aliquots of this sample were then treated with SGF, as in Example 4. The amount of protein remaining in the exines after 45 minutes, measured by UV-vis spectroscopy, was found to be 58% w/w of the original quantity of protein. The protein was released when treated with PBS as in Example 5.

### Example 10

### Eudragit® L-100/55/Ibuprofen as a Protective Additive

A mixture of a polymethacrylate polymer and ibuprofen was used as a protective additive for the Example 1 protein encapsulated in 25 μm exine shells.

A solution of 43.1 mg of the protein in 0.4 ml of water and 0.2 ml of acetone was added dropwise to 213.6 mg of the prepared exine shells, with gentle stirring, and the mixture left under vacuum for an hour. The sample was freeze dried to constant weight. A solution containing 68.1 mg of Eudragit® L-100/55 and 36.2 mg of ibuprofen in 0.5 ml of acetone-water (49:1) was then added dropwise to the protein-loaded exines, with gentle stirring, and the mixture was left under vacuum for an hour and then freeze dried to constant weight. This operation was repeated with a solution containing 68.1 mg of Eudragit® L-100/55 and 36.2 mg of ibuprofen in 0.5 ml of acetone-water (49:1), which was added dropwise to the protein-loaded exines with gentle stirring. The mixture was left under vacuum for an hour and then freeze dried to constant weight. The resultant exines contained 92.7 mg of the protein per gram of sample.

Aliquots of the sample were treated with SGF at 37° C., as in Example 4. The amount of protein remaining in the exines was measured after 45 minutes, by UV-vis spectroscopy, and found to be 67% w/w of the original quantity of protein. The protein was released when treated with PBS as in Example 5.

### Example 11

### Cod Liver Oil as a Protective Additive

A mixture of cod liver oil and 1% of lecithin was co-encapsulated as a protective additive with the protein used in Example 1, in 25 μm exine shells, using the following protocol.

A solution of 54.0 mg of the protein in 0.5 ml of water was added to 505.3 g of cod liver oil containing 1% lecithin. The mixture was stirred to afford a homogeneous emulsion. This emulsion was then added dropwise to 509.4 g of the prepared exine shells, with gentle stirring. The mixture was left under vacuum for an hour and then freeze dried to constant weight. The resultant exines contained 51.2 mg of the protein per gram of sample.

Aliquots of the sample were treated with SGF, as in Example 4. The amount of protein remaining in the exines after 45 minutes was measured by UV-vis spectroscopy to be 57% w/w of the original quantity of protein. This demonstrates that the oil/lecithin mixture can provide protection for 57% w/w of the co-encapsulated protein against gastric fluid.

### Example 12

### Eudragit® L-100/55 as a Protective Additive

Eudragit® L-100/55 was used as a protective additive for an exine-encapsulated protein of RMM ca. 22000. Two separate applications of the Eudragit® L-100/55 additive were effected to provide a double co-encapsulated layer.

A mixture of 3.9 mg in 0.5 ml of water and 29.4 mg of Eudragit® L-100/55 in 0.5 ml of ethanol was added dropwise to 110.3 mg of the prepared exine shells, with gentle stirring, and the mixture left under vacuum for an hour. The sample was dried over P₂O₅ to constant weight. A solution of 39.5 mg of Eudragit® L-100/55 in 0.3 ml of ethanol was then added dropwise to the protein-loaded exines, with gentle stirring, and the mixture was left under vacuum for an hour and then freeze dried to constant weight. The resultant exines contained 21.3 mg of the protein per gram of sample.

Aliquots of the sample were treated with SGF at 37° C., as in Example 4, and the amount of protein remaining in the exines was measured every 15 minutes by UV-vis spectroscopy.

After 45 minutes in SGF, 86% w/w of the original quantity of protein remained in the exines. This illustrates that two co-encapsulated protective layers of Eudragit® L-100/55 may be used to protect an exine-encapsulated protein from degradation in the stomach following oral administration. The protein was released when treated with PBS as in Example 5.

### Example 13

### Eudragit® L-100/55 as a Protective Additive

Here Eudragit® L-100/55 was used as a protective additive for an exine-encapsulated protein, the two being co-encapsulated sequentially into 25 μm exine shells. In this case the weight ratio of protective additive to active substance (protein) was nearly 3:1.

A solution of 42.1 mg of the protein used in Example 1, in 0.4 ml of water with a few drops of ethanol, was added dropwise to 505 mg of the prepared exine shells, with gentle stirring, and the mixture left under vacuum for an hour. The sample was dried over P₂O₅ to constant weight. A solution of 128.3 mg of Eudragit® L-100/55 in 0.7 ml of ethanol was then added dropwise to the protein-loaded exines, with gentle stirring. The mixture was left under vacuum for an hour and then dried over P₂O₅ to constant weight. The resultant exines contained 114.6 mg of the protein per gram of sample.

Aliquots of the sample were then treated with SGF at 37° C. as in Example 4, and the amount of protein remaining in the exines was measured every 15 minutes by UV-vis spectroscopy.

After 45 minutes in the SGF, 69% w/w of the original quantity of protein remained in the exines, thus illustrating that Eudragit® L-100/55 can be used to protect an exine-encapsulated protein from degradation in the harsh acidic conditions encountered in the stomach following oral administration. The protein was released when treated with PBS as in Example 5.

### Example 14

### Eudragit® L-100/55 as a Protective Additive

Eudragit® L-100/55 was again used as a protective additive for an exine-encapsulated protein, as in Example 7 with the two constituents being encapsulated sequentially into 25 μm exine shells. In this case the weight ratio of protective additive to protein was nearly 1:1.

A solution of 38.4 mg of the protein in 0.36 ml of water with a few drops of ethanol was added dropwise to 179.8 mg of the prepared exine shells, with gentle stirring. The mixture was left under vacuum for an hour and freeze dried to constant weight. A solution of 48.2 mg of Eudragit® L-100/55 in 0.7 ml of ethanol was then poured dropwise onto the protein-loaded exines, with gentle stirring, and the mixture was left under vacuum for an hour and then dried over P₂O₅ to constant weight. The resultant exines contained 144.1 mg of the protein per gram of sample.

Aliquots of the sample were then treated in SGF at 37° C. as in Example 4, and the amount of protein remaining in the exines was measured every 15 minutes by UV-vis spectroscopy. After 45 minutes in SGF, 55% w/w of the original quantity of protein remained in the exines. This illustrates that one co-encapsulated layer of Eudragit® L-100/55 may be used to protect an exine-encapsulated protein from degradation in the harsh acidic conditions encountered in the stomach following oral administration. Again the protein was released when treated with PBS as in Example 5.

### Example 15

### Gelatine and Eudragit® L-100/55 as Protective Additives

A mixture of gelatine and Eudragit® L-100/55 was used as a protective additive for the Example 1 protein encapsulated in 25 μm exine shells.

A solution of 40.8 mg of the protein and 17.2 mg of gelatine in 0.38 ml of water with a few drops of ethanol was added dropwise to 190.6 mg of the prepared exine shells, with gentle stirring, and the mixture left under vacuum for an hour. The sample was freeze dried to constant weight. A solution of 128.3 mg of Eudragit® L-100/55 in 0.7 ml of ethanol was then added dropwise to the protein-loaded exines, with gentle stirring. The mixture was left under vacuum for an hour and then dried over P₂O₅ to constant weight. The resultant exines contained 108.2 mg of the protein per gram of sample.

Aliquots of the sample were then immersed in SGF at 37° C., as in Example 4, and the amount of protein remaining in the exines was measured every 15 minutes by UV-vis spectroscopy.

After 45 minutes in SGF, 84% w/w of the original quantity of protein remained in the exines. This illustrates that Eudragit® L-100/55 and gelatine together may be used to protect an exine-encapsulated protein from degradation in the harsh acidic conditions encountered in the stomach following oral administration.

### Example 16

### Lauric Acid as a Protective Additive

Lauric acid was used as a protective additive for an exine-encapsulated protein, the two being encapsulated sequentially into 25 μm exine shells.

A solution of 53.1 mg of the Example 1 protein in 0.5 ml of water and 0.25 ml of acetone was added dropwise to 263.6 mg of the prepared exine shells, with gentle stirring, and the mixture left under vacuum for an hour. The sample was freeze dried to constant weight. A solution of 270.2 mg of lauric acid in 0.6 ml of ethanol was then added dropwise to the protein-loaded exines, with gentle stirring, and the mixture was left under vacuum for an hour and then freeze dried to constant weight. The resultant exines contained 90.3 mg of the protein per gram of sample.

Aliquots of the sample were then treated with SGF at 37° C., as in Example 4. The amount of protein remaining in the exines was measured after 45 minutes, by UV-vis spectroscopy, to be 19% w/w of the original quantity of protein. The protein was released when treated with PBS as in Example 5.

### Example 17

### Gelatine and Ibuprofen as Protective Additives

Gelatine and ibuprofen were used as protective additives for the Example 1 protein, the two being encapsulated sequentially into 25 μm exine shells.

A solution of 98.2 mg of the protein and 138.3 mg of gelatine in 0.9 ml of water with a few drops of ethanol was added dropwise to 548.2 mg of the prepared exine shells, with gentle stirring, and the mixture left under vacuum for an hour. The sample was dried over P₂O₅ to constant weight. A solution of 418.1 mg of ibuprofen in 0.45 ml of ethanol was then added dropwise to the protein-loaded exines, with gentle stirring, and the mixture left under vacuum for an hour and then dried to constant weight. The resultant exines contained 81.6 mg of the protein per gram of sample.

Aliquots of the sample were then treated with SGF at 37° C., as in Example 4, and the amount of protein remaining in the exines after 45 minutes, measured by UV-vis spectroscopy, was found to be 24% w/w of the original quantity of protein.

### Example 18

### Ibuprofen and Eudragit® L-100/55 as a Protective Additive

A mixture of ibuprofen and a polymethacrylate polymer was used as a protective additive for the Example 1 protein encapsulated in 25 μm exine shells.

A solution of 55.7 mg of the protein in 0.51 ml of water and 0.13 ml of ethanol was added dropwise to 303.1 mg of the prepared exine shells, with gentle stirring, and the mixture left under vacuum for an hour. The sample was freeze dried to constant weight. A solution containing 39.9 mg of Eudragit® L-100/55 and 314.9 mg of ibuprofen in 0.5 ml of ethanol was then added dropwise to the protein-loaded exines, with gentle stirring, and the mixture was left under vacuum for an hour and then freeze dried to constant weight. The resultant exines contained 78.0 mg of the protein per gram of sample.

Aliquots of the sample were treated with SGF at 37° C., as in Example 4. The amount of protein remaining in the exines was measured after 45 minutes, by UV-vis spectroscopy, and found to be 65% w/w of the original quantity of protein. The protein was released when treated with PBS as in Example 5.

### Example 19

### Palmitic Acid as a Protective Additive

In this example, palmitic acid was used as a protective additive for an exine-encapsulated protein, the two being encapsulated sequentially into 25 μm exine shells.

A solution of 57.1 mg of the Example 1 protein in 0.5 ml of water and 0.1 ml of ethanol was added dropwise to 327.6 mg of the prepared exine shells, with gentle stirring, and the mixture left under vacuum for an hour. The sample was freeze dried to constant weight. A solution of 321.8 mg of palmitic acid in 0.5 ml of ethanol/chloroform (1:1) was then added dropwise to the protein-loaded exines, with gentle stirring, and the mixture was left under vacuum for an hour and then dried to constant weight. The resultant sample contained 80.8 mg of the protein per gram of sample.

Aliquots of the sample were then treated with SGF at 37° C., as in Example 4. The amount of protein remaining in the exines was measured after 45 minutes, by UV-vis spectroscopy, and determined to be 14% w/w of the original quantity of protein.

### Example 20

### Aquacoat® CPD/Lauric Acid as a Protective Additive

A mixture of a cellulose-based polymer (Aquacoat® CPD) and the plasticiser lauric acid was used as a protective additive for the Example 1 protein encapsulated in 25 μm exine shells. Two separate applications of the Aquacoat® CPD/lauric acid additive were used.

A solution of 113.4 mg of the protein in a mixture of 0.5 ml of water and 0.5 ml of acetone was added to a solution of 71.4 mg of Aquacoat® CPD and 32.9 mg of lauric acid in 0.5 ml of acetone containing 2% water. The mixture was stirred to afford a homogeneous emulsion. This emulsion was then added dropwise to 543.3 mg of the prepared exine shells, with gentle stirring, and the mixture left under vacuum for an hour. The sample was freeze dried to constant weight. A solution containing a mixture of 142.8 mg Aquacoat® CPD and 65.7 mg of lauric acid in 1 ml acetone containing 2% water was then added dropwise to the protein-loaded exines, with gentle stirring. The mixture was left under vacuum for an hour and then freeze dried to constant weight. The exines contained 117.0 mg of the protein per gram of sample.

Aliquots of the sample were treated with SGF, as in Example 4. The amount of protein remaining in the exines after 45 minutes, as measured by UV-vis spectroscopy, was found to be 47% w/w of the original quantity of protein.

### Example 21

### Cocoa Butter as a Protective Additive

Ascorbic acid was used as the active substance and cocoa butter and lecithin as protective additives, the three being encapsulated together into 25 μm exine shells.

A solution of 525.5 mg of ascorbic acid in 1 ml of water was mixed with molten cocoa butter/lecithin (9:1) (578.3 mg) to afford an emulsion, and was added dropwise to 1.08 g of the prepared exines shells with gentle stirring. The mixture was left under vacuum for an hour. The sample was then dried over P₂O₅ to constant weight. The resultant exines contained 240.6 mg of the ascorbic acid per gram of sample.

Aliquots of the sample were treated with water at room temperature as in Example 1. The amount of the hydrophilic active substance remaining in the exines, measured after 45 minutes by UV-vis spectroscopy, was 25% w/w of the original quantity.

### Example 22

### Histoclear™ as Protective Additive and Cod Liver Oil as an Active Additive

A mixture of 2.01 g of cod liver oil and 507 mg of Histoclear™ II (a mixture of food-grade essential oils, including limonene and other terpenes (ex National Diagnostics, Hull, UK)) was added dropwise to 2.08 g of the prepared exine shells, with gentle stirring. The mixture was left (no vacuum) for an hour to afford a powder.

The smell and taste of the cod liver oil detectable after this co-encapsulation with Histoclear™ II was found to be less than when the oil alone was encapsulated in the exine shells. In addition, the co-encapsulation with Histoclear™ II produced a much more freely flowing powder than the oil and exines alone.

Thus an oil mixture such as Histoclear™ II may be used as an additive not only to protect a co-encapsulated active substance, but in cases to mask its taste and/or aroma, and/or to modify the physical form of the exine/active combination, possibly facilitating its subsequent formulation for example into a food, beverage or pharmaceutical product. It is unexpected that a liquid can be used as a protective additive in this way. Conventionally, solid external coatings would have been applied to protect an encapsulated active substance, but by co-encapsulating an additive in accordance with the present invention, protection can be achieved using a wider range of materials. This illustrates yet further the broad potential of the present invention.

The examples above show that all of the additives tested were capable of providing at least a degree of protection, against the low pH SGF and/or against hydrophilic conditions, for a co-encapsulated active substance. All could therefore be used in a formulation according to the invention, to protect an orally delivered active substance so as to allow it to reach its intended destination for example in the bloodstream or in the gastro-intestinal tract. They could also be used to prevent or otherwise control the release of an active substance through a porous exine shell delivery vehicle in a formulation intended for instance for topical or respiratory delivery.

The Eudragit® L-100/55 proved a particularly effective protectant, especially in combination with a fatty acid. Impregnating the exine shells twice with either the same additive or two different additives also appeared to improve protection of the active substance against the simulated gastric fluid.

### Example 23

### Pharmaceutical or Dietetic Formulation

A pharmaceutical or dietetic formulation may be prepared, according to the present invention, using spore-derived exine shells for instance as prepared in the examples above, and loading them with both a pharmaceutical or dietetic substance such as a protein and one or more protective additives. The loaded exine shells may then be suspended in any suitable vehicle, for example a vehicle suitable for oral administration, or may be otherwise formulated for example into tablets or capsules. The additive(s) contained within the exine shells will help to protect the co-encapsulated active substance from degradation in the harsh acidic environment of the stomach, allowing it to reach its intended site of action which may for instance be the gut (for example for food supplements such as probiotics) or the bloodstream (for example for a hormone such as insulin).

A similar formulation may be prepared for use as, or as part of, a food product (including a beverage), a supplemented food product or a food supplement.

### Example 24

### Starch as a Protective Additive

In this example a protein of relative molecular mass (RMM) ca. 6000 was co-encapsulated in 25 μm exine shells with a mixture of starch and 10% of glycerol as the protective additive. The following procedure was used: —

A solution of 27.5 mg of the protein in 0.2 ml of water 0.2 ml of 2M-HCl and 0.02 ml of Histoclear™ was added to a solution 681.8 mg of starch (glycerol 10%) in water (18.2 g of starch and 1.8 g of glycerol in 50 ml of water). The mixture was stirred and 316.5 mg of the prepared exine shells were added to this mixture, with gentle stirring. The mixture was left under vacuum for an hour and then freeze dried to constant weight. The resultant exines contained 19.3 mg of the protein per gram of sample.

Aliquots of the sample were treated with SGF, as in Example 4. The amount of protein remaining in the exines after 5 and 45 minutes was measured by UV-vis spectroscopy to be 29% and 10% w/w of the original quantity of protein, respectively. This demonstrates that starch can provide protection of the co-encapsulated protein against gastric fluid.

1-37. (canceled)

38. A formulation comprising an active substance encapsulated within an exine shell of a naturally occurring spore, together with a protective additive which is also encapsulated within the exine shell, wherein the active substance comprises a hydrophilic and/or hydrolysable and/or acid-labile substance and/or a proteinaceous material; a carbohydrate; a lipid; a nucleoside, nucleotide or nucleic acid; a vitamin or co-vitamin; an essential fatty acid; an essential mineral or mineral-containing substance; a glyconutrient; a phytonutrient; a nutritional agent; or a micro-organism; and/or a substance selected from peptides, enzymes, probiotics and prebiotics; and the protective additive is a solid or semi-solid at room temperature.

39. The formulation of claim 38, wherein the active substance comprises a hydrophilic and/or hydrolysable and/or acid-labile substance and/or a substance selected from peptides, enzymes, probiotics and prebiotics.

40. The formulation of claim 38, wherein the active substance comprises a substance selected from peptides, enzymes, probiotics and prebiotics.

41. The formulation of claim 38, wherein the protective additive is water insoluble.

42. The formulation of claim 38, wherein the protective additive comprises one or more lipids or materials having a lipid component.

43. The formulation of claim 42, wherein the protective additive comprises a lipid or material having a lipid component selected from the group consisting of a butter or other solid fat; a fatty acid having a C11 to C22 carbon chain length; a fatty acid ester; a fatty acid amide; a phospholipid; a glycolipid; a lipid sulphate; a lipid sulphonate; a monoglyceride; a diglyceride, and a triglyceride.

44. The formulation of claim 43, wherein the protective additive comprises a fatty acid ester.

45. The formulation of claim 43, wherein the protective additive comprises a fatty acid selected from the group consisting of lauric acid, myristic acid, palmitic acid, stearic acid, behenic acid, sebacic acid, undecanedioic acid, 1,10-decanedicarboxylic acid, brassylic acid, 1,12-dodecanedicarboxylic acid and 1,15-pentadecanedioic acid.

46. The formulation of claim 43, wherein the protective additive comprises a butter or solid fat selected from the group consisting of cocoa butter and hardened palm kernel oil.

47. The formulation of claim 42, wherein the protective additive comprises lecithin.

48. The formulation of claim 42, wherein the protective additive comprises a material having a lipid component selected from the group consisting of benzoic acid, 4-isopropylbenzoic acid, palmitoyl ascorbic acid and SULINDAC™.

49. The formulation of claim 38, wherein two or more layers of protective additive are encapsulated in the exine shell.

50. The formulation of claim 38, wherein the protective additive increases the duration of retention of the active substance within the exine shell, as compared to the retention of active substance without the co-encapsulated protective additive by at least 1.5-fold.

51. The formulation of claim 38, wherein the protective additive forms a protective layer around the active substance.

52. The formulation of claim 38, wherein the weight ratio of the active substance to the protective additive within the exine shell is from 10:1 to 0.01:1.

53. The formulation of claim 38, wherein the protective additive prevents or delays the release of the active substance until the exine shell reaches its intended site of action, wherein the site of action is selected from the mouth, gut, bloodstream, gastro-intestinal tract, intestine, upper intestinal tract and lower intestinal tract.

54. The formulation of claim 38, wherein the protective additive prevents or delays the release of the active substance until the exine shell reaches its intended site of action, wherein the site of action is selected from skin or hair.

55. The formulation of claim 38, wherein the active substance is suitable and/or intended and/or adapted for oral, buccal, nasal, pulmonary, intravenous, intra-muscular, topical, transdermal, subcutaneous, intraperitoneal, vaginal, rectal or colonic delivery, or for delivery into the eye or ear.
